SEQUENCE LISTING (15 sequences)

SEQ ID NO: 1 - protein, 74 aa, Hermetia illucens
MQIFVKTLTGKTITLEVEPSDTIENVKAKIQDKEGIPPDQQRLIFAGKQLEDGRTLSDYNIQKESTLHLVLRLR

SEQ ID NO: 2 - protein, 13 aa, Hermetia illucens
VALTGLTVAEYFR

SEQ ID NO: 3 - protein, 22 aa, Hermetia illucens
ARTKQTARKSTGGKAPRKQLAT

SEQ ID NO: 4 - protein, 63 aa, Hermetia illucens
RGFRKHFNNLPICVEGLAGDIGSILLGVGSDIGALAGAIANLALIAGECAAQGEAGAAVVAAT

SEQ ID NO: 5 - protein, 169 aa, Hermetia illucens
MASKFLGNPNHNIGGGVFAAGNTRSNTPSLGAFGTLNLKDHSLGVSHTITPGVSDTFSQNARLNILKTPDHRVDANVFNSHTRLNNGFAFDKRGGSLDYTHRAGHGLSLGASHIPKFGTTAELTGKANLWRSPSGLSTFDLTGSASRTFGGPMAGRNNFGAGLGFSHRF

SEQ ID NO: 6 - protein, 17 aa, Hermetia illucens
MWITNGGVANWYFVLAR

SEQ ID NO: 7 - protein, 12 aa, Hermetia illucens
AERVGAGAPVYL

SEQ ID NO: 8 - protein, 38 aa, Hermetia illucens
MAGGKAGKDSGKAKAKAVSRSARAGLQFPVGRIHRHLK

SEQ ID NO: 9 - protein, 103 aa, Hermetia illucens
MTGRGKGGKGLGKGGAKRHRKVLRDNIQGITKPAIRRLARRGGVKRISGLIYEETRGVLKVFLENVIRDAVTYTEHAKRKTVTAMDVVYALKRQGRTLYGFGG

SEQ ID NO: 10 - protein, 6 aa, Hermetia illucens
WKWKWK

SEQ ID NO: 11 - protein, 9 aa, Hermetia illucens
CHPSTHRYC

SEQ ID NO: 12 - protein, 13 aa, Hermetia illucens
KGLSGPSEWWVWV

SEQ ID NO: 13 - protein, 11 aa, Hermetia illucens
KVHGSLARAGK

SEQ ID NO: 14 - protein, 16 aa, Hermetia illucens
RQIKIWFQNRRMKWKK

SEQ ID NO: 15 - protein, 9 aa, Hermetia illucens
EPRTPWDWV

(19) United States
(12) Patent Application Publication
(10) Pub. No.: US 2026/0355278 A1
(43) Pub. Date: Nov. 12, 2026

(54) BLACK SOLDIER FLY PROTEIN EXTRACTS

(71) Applicant: NeoManna Ltd, Tel-Aviv (IL)

(72) Inventors: Gal WITTENBERG, ModiIn (IL); Matan LEVY, Rehovot (IL); Miri LAPIDOT, Lehavim (IL); Amir BARZILAY, Kochav Yair (IL); Bar BEN SHITRIT, Rehovot (IL); Phillip SHAFIR, Rehovot (IL)

(73) Assignee: NeoManna Ltd, Tel-Aviv (IL)

(21) Appl. No.: 19/487,005

(22) PCT Filed: May 22, 2024
(86) PCT No.: PCT/IL2024/050501

§ 371 Date: Nov. 24, 2025

Related U.S. Application Data
(60) Provisional application No. 63/468,037, filed on May 22, 2023.
(60) Provisional application No. 63/621,156, filed on Jan. 16, 2024.

(51) Int. Cl. A61K 38/17; A61K 38/01; A61P 29/00; C07K 7/06; C07K 7/08; C07K 14/435; C12P 21/06

(52) U.S. Cl. CPC A61K 38/1767; A61K 38/012; A61P 29/00; C07K 7/06; C07K 7/08; C07K 14/43577; C12P 21/06

(57) ABSTRACT

A black soldier fly (BSF) protein extract comprising at least one peptide consisting of the amino acid sequence selected from the group consisting of SEQ ID NO: 2-15. Methods of solubilized BSF proteins and uses thereof are also disclosed.

### RELATED APPLICATIONS

This application claims the benefit of priority of U.S. Provisional Patent Application No. 63/468,037 filed on May 22, 2023 and U.S. Provisional Patent Application No. 63/621,156 filed on Jan. 16, 2024, the contents of which are incorporated herein by reference in their entirety.

### SEQUENCE LISTING STATEMENT

The XML file, entitled 99460 Sequence Listing.xml, created on May 22, 2024, comprising 13,892 bytes, submitted concurrently with the filing of this application is incorporated herein by reference.

### FIELD AND BACKGROUND OF THE INVENTION

The present invention, in some embodiments thereof, relates to black soldier fly (BSF) protein extracts and uses thereof.

The Food and Agriculture Organization of the United Nations predicts that the global population will increase to 9 billion by 2050, with global meat consumption projected to increase as a result. A large amount of feed is required to meet this increase in animal meat consumption. Most protein resources added to feed are highly dependent on imports, and the price of protein resources has been steadily rising in recent years. Protein feed resources such as soybean schlegelii and fishmeal are expensive, and some, such as fishmeal, are non-sustainable. Therefore, resources are needed to replace them.

Black soldier fly (BSF), Hermetia illucens (Diptera: Stratiomyidae), is a warm-climate and innocuous insect that rapidly colonizes decomposing waste substances, such as food scraps or kitchen waste, straw, manure, etc. The variety of substances and efficiency of their consumption by BSF larvae is higher than that by Drosophila melanogaster, Apis mellifera, and Bombyx mori. Habitats of BSF larvae are characterized by abundance of various microorganisms. BSF is known to live in adverse environments, indicating that BSF has an innate immune system that can cope with a wide variety of pathogens, by producing various substances, such as peptides [Choi, W. H.; Choi, H. J.; Goo, T. W.; Quan, F. S. Novel antibacterial peptides induced by probiotics in Hermetia illucens (Diptera: Stratiomyidae) larvae. Entomol. Res. 2018, 48, 237-247.], that protect against bacteria, fungi, and viruses. A defensin-like peptide 4 (DLP4) was recently detected and extracted from various immune tissues of BSF larvae. The survival rate of mice infected with methicillin-resistant Staphylococcus aureus (MRSA) treated with DLP4 was 80-100% [Li, Z et al Sci. Rep. 2017, 7, 12124.]. This finding demonstrated that DLP4 is a promising novel candidate antimicrobial peptide (AMP) against MRSA infections.

BSF protein digestion using non-specific proteases such as alkaline protease is disclosed in the following articles: (Lu J, 2022), (Batish I, 2020), (Firmanseyah M, 2019), (Triani R, 2021) (Mouithys-Mickalad A, 2021).

Additional background art includes Bose et al., Journal of Proteomics 269 (2022) 104724 and PCT Applications WO 2021/054823 and WO 2021/054824.

### SUMMARY OF THE INVENTION

According to an aspect of the present invention, there is provided a black soldier fly (BSF) protein extract comprising at least one peptide consisting of the amino acid sequence selected from the group consisting of SEQ ID NO: 2-15.

According to an aspect of the present invention, there is provided a black soldier fly (BSF) protein extract being enriched or supplemented with at least one peptide consisting of the amino acid sequence selected from the group consisting of SEQ ID NO: 1-15.

According to embodiments of the invention, the extract is devoid of peptides of less than 6 amino acids.

According to embodiments of the invention, the at least one peptide is formulated for oral delivery.

According to embodiments of the invention, the at least one peptide is encapsulated.

According to embodiments of the invention, the at least one peptide is modified by a method selected from the group consisting of lipidization, cationization and PEGylation.

According to embodiments of the invention, the at least one peptide is cyclized.

According to embodiments of the invention, the extract further comprises an absorption enhancer or a mucolytic agent.

According to another aspect, there is provided an animal feed or animal feed supplement comprising at least one isolated peptide consisting of the amino acid sequence selected from the group consisting of SEQ ID NO: 2-15.

According to embodiments of the invention, the animal feed or animal feed supplement is a livestock food, an aquaculture food or a companion animal food.

According to embodiments of the invention, the animal feed or animal feed supplement further comprises plant biomass.

According to embodiments of the invention, the plant biomass comprises a vegetable biomass.

According to another aspect, there is provided a method of preparing a hydrolyzed BSF protein extract comprising:
contacting a protein-solubilized BSF protein extract with:
   (i) a hydrolyzing enzyme selected from the group consisting of ubiquitin specific peptidase 5 (USP5), ubiquitin specific peptidase 4 (USP4) and ubiquitin specific peptidase 15 (USP15) under conditions that allow the enzyme to hydrolyze the BSF extract;
   (ii) a combination of trypsin and arginine-C protease under conditions that allow the trypsin and arginine-C protease to hydrolyze the BSF extract, thereby preparing the hydrolyzed BSF protein extract; or
   (iii) Glu-N protease under conditions that allow the Glu-N protease to hydrolyze the BSF extract.

According to embodiments of the invention, the protein-solubilized BSF protein extract is generated from BSF larvae.

According to embodiments of the invention, the larvae are between 2-40 days of age post hatching.

According to embodiments of the invention, the method further comprises generating the protein-solubilized BSF protein extract by:
(a) defatting BSF or BSF larvae thereof to generate a defatted extract of BSF or BSF larvae;
(b) solubilizing the defatted extract.

According to embodiments of the invention, the solubilizing is effect in a buffer, a solvent or water.

According to embodiments of the invention, the buffer comprises Tris-HCl.

According to embodiments of the invention, the BSF or BSF larvae are dried prior to defatting.

According to embodiments of the invention, the method further comprises generating a pulp from the BSF or BSF larvae prior to the defatting.

According to embodiments of the invention, the method further comprises separating a water-soluble fraction of the pulp from solid material of the pulp following the defatting.

According to embodiments of the invention, the method further comprises drying the pulp prior to said solubilizing.

According to embodiments of the invention, the defatting is effected by at least one of the separating means selected from the group consisting of decantation, tricantation, centrifugation and pressing.

According to another aspect there is provided a hydrolyzed BSF protein extract obtainable according to the methods described herein.

According to another aspect there is provided a method of solubilizing BSF proteins comprising:
contacting BSF proteins with about 100 mM Tris-HCl buffer at a ratio of no more than 20% (w/v), under conditions that promote solubilization of the BSF proteins, thereby solubilizing the BSF proteins.

According to embodiments of the invention, the BSF proteins are extracted from dried BSF or BSF larvae.

According to embodiments of the invention, the BSF proteins are extracted from a wet pulp comprising BSF or BSF larvae.

According to embodiments of the invention, the BSF proteins are obtained by removing lipids from BSF larvae or BSFs under wet conditions.

According to embodiments of the invention, the BSF proteins are obtained by removing lipids from BSF larvae or BSFs under dry conditions.

According to embodiments of the invention, the conditions comprise pH 7.5 for at least one hour.

According to embodiments of the invention, the method further comprises hydrolyzing said solubilized proteins.

According to embodiments of the invention, the hydrolyzing is effected by contacting said solubilized proteins with an enzyme selected from the group consisting of trypsin, ubiquitin specific peptidase 5 (USP5), ubiquitin specific peptidase 4 (USP4) and ubiquitin specific peptidase 15 (USP15), arginine-C protease and Glu-N protease.

According to embodiments of the invention, the hydrolyzing is effected by contacting said solubilized proteins with:
(i) a hydrolyzing enzyme consisting of trypsin under conditions that allow said enzyme to hydrolyze solubilized BSF proteins;
(ii) a hydrolyzing enzyme selected from the group consisting of ubiquitin specific peptidase 5 (USP5), ubiquitin specific peptidase 4 (USP4) and ubiquitin specific peptidase 15 (USP15) under conditions that allow said enzyme to hydrolyze said solubilized BSF proteins; or
(iii) a combination of trypsin and arginine-C protease under conditions that allow said trypsin and arginine-C protease to hydrolyze solubilized BSF proteins.

According to another aspect there is provided a solubilized BSF protein extract generated according to the methods described herein.

According to another aspect there is provided a composition comprising a BSF protein extract for use in treating or preventing an inflammatory disease.

According to embodiments of the invention, the BSF protein extract comprises the solubilized extract described herein.

According to embodiments of the invention, the BSF protein extract is obtained by removing lipids from BSF larvae or BSFs under wet conditions.

According to embodiments of the invention, the BSF protein extract is obtained by removing lipids from BSF larvae or BSFs under wet conditions.

According to embodiments of the invention, the composition is encapsulated.

According to yet another aspect there is provided a method of treating or preventing an inflammatory disease in a subject comprising providing a therapeutically effective amount of the BSF protein extract to the subject, thereby treating or preventing the inflammatory disease of the subject.

According to embodiments of the invention, the BSF protein extract is the extract described herein, the animal feed described herein, the hydrolyzed BSF protein extract described herein or the solubilized BSF protein extract described herein

According to embodiments of the invention, the BSF protein extract is obtained by removing lipids from BSF larvae or BSFs under wet conditions.

According to embodiments of the invention, the BSF protein extract is obtained by removing lipids from BSF larvae or BSFs under dry conditions.

According to embodiments of the invention, the subject is a human subject.

According to embodiments of the invention, the subject is a livestock animal.

According to embodiments of the invention, the subject is a companion animal.

According to embodiments of the invention, the subject is a fish or a crustacean.

According to embodiments of the invention, the inflammatory disease is IBD, Celiac, Crohn's disease or Colitis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph illustrating the effect of different methods of protein and peptide extraction on the amount of extracted protein into the buffer. BSF meal was solubilized in buffer at 5% w/w BSF meal:buffer. The supernatant was removed and the pellet was weighed. The % of the mass extracted was determined as the % mass that was removed by the buffer.

FIG. 2 is a photograph of a Coomassie-stained gel of BSF proteins solubilized according to embodiments of the invention.

FIGS. 3A-B are bar graphs illustrating the effect of stimuli on the secretion of cytokines that are known markers for inflammation from PBMCs in response to the protein fraction originating from a dry process (BSF meal preparation 1).

FIGS. 4A-B are a bar graphs illustrating the effect of stimuli on the secretion of cytokines that are known markers for inflammation from PBMCs in response to the protein fraction originating from the dry process (BSF meal preparation 2).

FIGS. 5A-B: are bar graphs illustrating the effect of stimuli on the secretion of cytokines that are known markers for inflammation from PBMCs in response to the dried and solubilized fractions generated during BSF wet processing (as described in Example 3).

FIGS. 6A-B: are bar graphs illustrating the effect of stimuli on the secretion of cytokines that are known markers for inflammation from PBMCs in response to sticky water produced during BSF wet processing (as described in Example 3).

### DESCRIPTION OF SPECIFIC EMBODIMENTS OF THE INVENTION

The present invention, in some embodiments thereof, relates to BSF protein extracts and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Due to an increasing world population, there is an urgent need for sustainable protein. Black soldier fly larvae (BSFL) are emerging as an alternative source for both food and animal feed, since they contain approximatively 42% crude proteins.

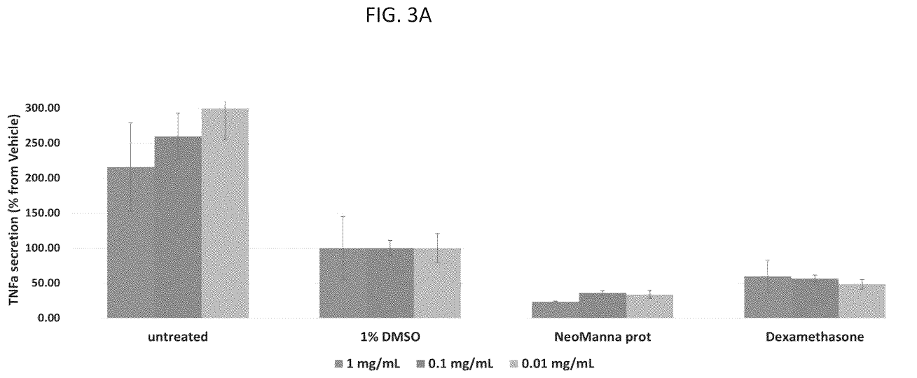

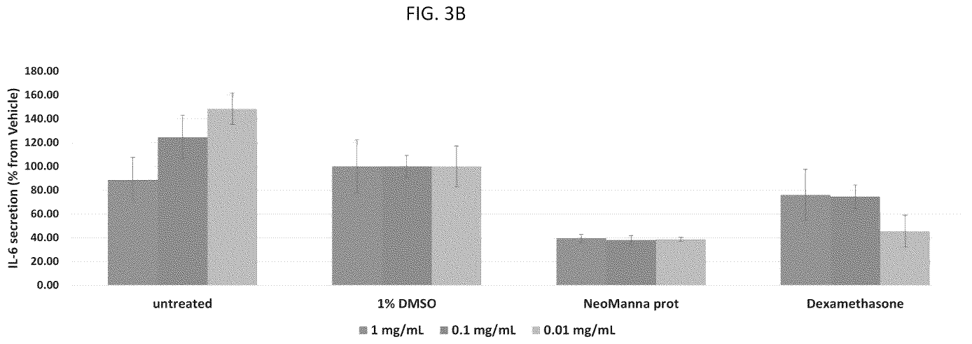

The present inventors have now discovered that when proteins are extracted from BSF larvae under particular conditions, the extract has anti-inflammatory properties. As illustrated in FIGS. 3A-B, 4A-B, 5A-B and 6A-B, the amount of inflammatory cytokines (e.g. IL-6 and TNFα) secreted by peripheral blood mononuclear cells (PBMCs) following contact with the BSF extract is much lower as compared to dexamethasone treated or control treated cells.

The present inventors have further uncovered potential anti-microbial peptides which may be released from the protein extract using hydrolyzing enzymes. Use of this hydrolyzed extract may be of particular significance for maintaining the health of both companion animals and livestock animals (including monogastric and ruminants) and/or for treating such animals with anti-microbial and/or anti-inflammatory diseases, thereby reducing the need for veterinary medicine.

Thus, according to an aspect of the invention there is provided a black soldier fly (BSF) protein extract for use in treating an inflammatory disorder.

In one embodiment, the extract comprises at least one peptide consisting of the amino acid sequence selected from the group consisting of SEQ ID NO: 2-15.

The term “protein extract” refers to an extract derived from the black soldier fly (or larvae thereof) which is enriched in BSF proteins and/or peptides as compared to the naturally occurring amount of proteins present in the fly or larvae. In one embodiment, the protein extract comprises less than half the amount of lipids as compared to the naturally occurring amount of lipids present in the fly or larvae. In another embodiment, the protein extract is essentially devoid of lipids.

In one embodiment, the protein extract is derived from the black soldier fly larvae which are harvested between 2-40 days post hatching, for example between 8-40 days post hatching, for example between 6 and 30 days post hatching, preferably between 6 and 20 days post hatching, more preferably 6-14 days post hatching, more preferably between 8-12 days post hatching, e.g. 10 days post hatching. According to a particular embodiment, the extract is derived from larvae which are harvested 12 hours-3 days before the larvae transform into prepupae, such as 1-2 days before transformation.

BSF (or larvae thereof) may be killed by blenching, for example by exposing them to hot water at a temperature above 90 °C for 0.5-10 minutes, or in another example by exposing them to steam for 0.5-10 minutes.

According to a particular embodiment, the protein extract is at least partially devoid of (or substantially free of) lipids. Thus, the protein extract may be a pulp, from which lipids have been at least partially removed by a physical separation method, for example centrifugation or a mechanical press.

Optionally, prior to generation of the extract, the flies (or larvae thereof) may be rinsed. They may also be subjected to a steaming process.

In one embodiment, the separation of the fat fraction and the protein fraction is carried out under dry conditions. In one example, drying of larvae may be carried out by exposing them to hot air for 1-60 minutes, more specifically 5-30 minutes, at air temperature of 80-250 °C, more specifically 100-200 °C, more specifically 125-175 °C. In another example the BSFL may be dried in a drying room or chamber with air temperature of 50-150 °C, more specifically 70-110 °C, for a period of 1-48 hours, more specifically 12-36 hours, even more specifically 20-30 hours. In still another embodiment, BSFL may be dried using a drier, such as a microwave drier or a fluidized bed drier, in which the temperature is set to 80-200 °C.

The flies or larvae are typically reduced in size by mechanical treatment e.g. crushed, homogenized or minced under conditions whereby crude fat (i.e. lipids) can be extracted therefrom (squeezed or pressed therefrom). Such crushing and mechanical force can be achieved, for example, using an expeller. The defatted dry matter is referred to as defatted BSFL protein meal.

In one embodiment, the separation of the fat fraction and the protein fraction is carried out under wet conditions. Following killing, as described herein above, BSFL may be reduced in size, for example by crushing or mincing, reaching a particle size between 5-1000 microns, resulting in a BSFL pulp. In one example, BSFL may be heated to a temperature of 75-99 degrees for 0.5-3 minutes, before moving on to a physical separation step. In another example, physical separation is performed without prior heating. The pulp or heated pulp is separated by physical means, for example by decantation and/or tricantation and/or centrifugation and/or pressing, separating the oil from solid mass and from the water-soluble fractions. Both the water-soluble fraction (also referred to herein as the sticky water fraction) and the solid mass (also referred to herein as protein pulp) contain proteins and peptides. In one example, the water-soluble fraction and the solid mass are mixed to create a watery protein pulp.

In one embodiment, the protein fraction (e.g. watery protein pulp) is then dried. Watery pulp drying can be performed by heating to 70-160 degrees, until reaching water content<6%, yielding BSFL defatted total protein meal. In another example, the solid mass and the water fraction are kept separate. In this example, the solid mass is dried by heating to 70-160 degrees to a water content of <6%, yielding BSFL defatted solid protein meal. In one example, the water-soluble fraction is used as is following separation. In another example, the water-soluble fraction is heated to 70-100 degrees for 0.5-15 minutes, prior to being used, yielding heat treated water-soluble BSFL protein. In a third example, the water-soluble fraction is concentrated and/or dried prior to use. Drying and/or concentration is performed by one or a combination of several methods, including, but not limited to falling film evaporation, ultrafiltration, reverse osmosis, heating, and spray drying, yielding concentrated or dried water-soluble BSFL proteins.

Protein in the crude protein extract may be solubilized and optionally further enriched using a number of protein extraction protocols-see for example Bose et al., Journal of Proteomics 269 (2022) 104724, the contents of which are incorporated herein by reference.

Defatted BSF larvae protein is commercially available—for example from Zemach Taarovot (Tzemach, Israel).

Exemplary protein extraction buffers which may be used for solubilization of BSF proteins from crude protein extracts include Tris based buffers—e.g. Tris-HCl (e.g. between 75-300 mM, 75-150 mM, e.g. about 100 mM) at a pH between 7-8 (e.g. about 7.5). Typically dry, defatted BSF protein is added to the buffer at a ratio of 5-35% w/w for 30 minutes-2 hours. In a particular embodiment, the dry, defatted BSF protein is added to the buffer at a ratio of 5-20% w/w for 30 minutes-2 hours. In one embodiment, the ratio of dry defatted BSF protein:Tris buffer is no more than 20% w/w. According to an exemplary embodiment, the ratio is about 20%. Thus, for example, an exemplary method for solubilizing BSF proteins includes incubation in 100 mM Tris-HCl buffer at a pH of 7.5 and at a ratio of 20% w/w for one hour at room temperature.

In one embodiment, the BSF proteins are solubilized is water (e.g. tap water or distilled water).

In still another embodiment, the BSF proteins are solubilized in a solvent (e.g. DMSO).

As mentioned, in one embodiment, the BSF protein extract comprises at least one peptide consisting of the amino acid sequence as set forth in SEQ ID NOs: 2-15.

According to another embodiment, the BSF protein extract comprises at least one peptide consisting of the amino acid sequence as set forth in SEQ ID NO: 1.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2—), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino | Xaa | X |
| acid as above | | |

TABLE 2

| Non-conventional amino | | Non-conventional amino | |
|---|---|---|---|
| acid | Code | acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3- | Narg |
| | | guanidinopropyl)glycine | |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α- | Nmaib |
| | | aminoisobutyrate | |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclization does not severely interfere with peptide characteristics, cyclic forms of the peptide can also be utilized.

The peptides of some embodiments of the invention may be synthesized by any techniques that are known to those skilled in the art of peptide synthesis. Once synthesized, the peptides may be supplemented in the BSF protein extract. Thus, the present inventors contemplate a black soldier fly (BSF) protein extract being supplemented with at least one peptide consisting of the amino acid sequence selected from the group consisting of SEQ ID NO: 1-15.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A particular method of preparing the peptide compounds of some embodiments of the invention involves solid phase peptide synthesis.

Large scale peptide synthesis is described by Andersson Biopolymers 2000; 55 (3): 227-50.

According to another embodiment, the peptides are products of an enzyme reaction carried out on a solubilized extract of BSF proteins. Thus, the present inventors contemplate contacting a solubilized extract of BSF proteins with at least one peptidase including arginine-C protease, trypsin, leucyl amino-peptidase, endoproteinase Asp-N, papain, pronase, serine protease, alcalase, endoproteinase Glu-C, subtilisin, subtilisin A, a-chymotrypsin, cathepsin III, esperase, neutrase, pepsin, thermolysin and ubiquitin specific peptidase.

In order to release the disclosed peptides from a BSF protein extract particular combinations of hydrolyzing enzymes may be used.

For example, to release the peptide having an amino acid sequence as set forth in SEQ ID NO: 1 from the protein extract, the present inventors contemplate using trypsin.

To release the peptide having an amino acid sequence as set forth in SEQ ID NO: 2 from the protein extract, the present inventors contemplate using at least one of the following enzymes: ubiquitin specific peptidase 5 (USP5), ubiquitin specific peptidase 4 (USP4) and ubiquitin specific peptidase 15 (USP15);

To release the peptide having an amino acid sequence as set forth in SEQ ID NO: 3 from the protein extract, the present inventors contemplate using trypsin and arginine-C protease to hydrolyze.

Typically, the hydrolyzing enzyme is present in the method of the invention in an amount suitable for fast and efficient protein hydrolysis, wherein the water-soluble BSF protein is provided at a concentration of 2%-25% by weight based on the total weight of the protein composition, preferably 3.5%-20%, such as about 5%-20%.

The amount of hydrolyzing enzyme in the protein mixture may be between 0.005% and 3% by weight of the total weight of the protein mixture, preferably between 0.01% and 2%, more preferably between 0.015% and 5%, most preferably between 0.02% and 1%.

Preferably, the content of free amino acid residues in the hydrolyzed protein is less than 25%, less than 15%, less than 10%, less than 5%, less than 1% by weight based on the total weight of the protein subjected to the hydrolysis step in the method of the invention.

In one embodiment, the hydrolyzed protein extract is substantially devoid of peptides of less than 6 amino acids, less than 5 amino acids, less than 4 amino acids, less than 3 amino acids, less than 2 amino acids, or free amino acids.

In one embodiment, the hydrolyzed protein extract comprises peptides being greater than 6 amino acids.

In another embodiment, less than 30%, 20% or 10% of the protein in the extract is peptides of less than 6 amino acids, less than 5 amino acids, less than 4 amino acids, less than 3 amino acids, less than 2 amino acids, or free amino acids.

Following sufficient time for enzymatic hydrolysis to be completed, the reaction may be terminated by heating for a duration of between 30 seconds and 30 minutes, preferably between 45 seconds and 20 minutes, more preferably between 1 minute and 15 minutes or between 2 minutes and 10 minutes.

An embodiment is the method according to the invention, wherein in step d) the enzymatic hydrolysis in the hydrolyzed protein/peptidase solution of step c) is terminated by heating the hydrolyzed protein/peptidase solution to a temperature of 80 °C-105 °C, preferably 85 °C-103 °C, more preferably 90 °C-° 100 C. Typically, the enzyme inactivation step takes no more than two hour, e.g. between 2-10 minutes.

Once, hydrolyzed, the present inventors further contemplate enriching the extract so that there is a greater amount of one of the peptides in the extract as compared to a non-enriched extract. Thus, the present inventors contemplate BSF protein extracts being enriched with at least one peptide consisting of the amino acid sequence selected from the group consisting of SEQ ID NO: 1-15.

According to another aspect of the present invention there is provided a solubilized protein extract generated by contacting BSF proteins with between 75-150 mM Tris-HCl buffer at a ratio of no more than 35%, for example no more than 20% (w/w), under conditions that promote solubilization of the BSF proteins, thereby solubilizing the BSF proteins. The method for solubilizing BSF proteins has been described herein above, including exemplary conditions which include for example a pH of about 7.5 for at least one hour and/or the use of tap water or distilled water.

Optionally, the solubilized (and optionally hydrolyzed) extract described herein, may be dried, for example by applying any one or more of spray-drying, drying by fluidized bed drying, lyophilisation, refractive drying or spray drying. The benefits of providing dried water-soluble BSF protein/peptide compositions, are for example related to extended shelf life when stock piled, and to the opportunity to prepare an aqueous water soluble BSF protein/peptide composition at a selected final protein concentration in the aqueous solution, such that the protein concentration is controllable. Also the type of aqueous or non-aqueous solution for dissolving the protein can be selected, as well as the desired pH.

Optionally, the solubilized (and optionally hydrolyzed) extract described herein is concentrated, preferably concentrated such that the dry matter content of the concentrated enzymatically-hydrolyzed, water-soluble BSF protein/peptide is at least 30% by weight, preferably at least 50% by weight, based on the total weight of the concentrated enzymatically hydrolyzed water soluble BSF protein/peptide after the concentration step.

The BSF protein/peptide extracts described herein may be formulated for oral administration. The extracts may be encapsulated or non-encapsulated. The BSF protein/peptide extracts described herein may be added to companion animal food, human food stuff, livestock feed, aquaculture feed, etc. Thus, the protein extract may be considered as a food ingredient, supplement or additive, a companion animal food ingredient, supplement or additive, an animal feed ingredient, supplement or additive. Further, the protein extract may be considered as a human health therapeutic product, a companion animal health therapeutic product, a livestock animal health therapeutic product and an aquaculture health product. The term “feed” or “food” relates to both dry food, dry feed, liquid food, liquid feed and drinking water. The term “feed supplement” relates to an agent which is supplemented to a food or feed providing an added benefit (e.g. health benefit).

Thus according to another aspect of the present invention there is provided an animal feed ingredient or animal feed supplement, additive or health product comprising at least one isolated peptide consisting of the amino acid sequence selected from the group consisting of SEQ ID NO: 2-15.

According to another aspect there is provided an animal feed or animal feed supplement comprising a protein extract generated by contacting BSF proteins with between 75-150 mM Tris-HCl buffer at a ratio of no more than 20% (w/w), under conditions that promote solubilization of the BSF proteins, thereby solubilizing the BSF proteins. The method for solubilizing BSF proteins has been described herein above, including exemplary conditions which include for example a pH of about 7.5 for at least one hour.

The animal feeds, or feed supplement or food composition may comprise additional edible materials including for example crude protein, crude fat, carbohydrates, polysaccharides, starch, crude fibers, ash, minerals, trace elements, vitamins, fatty acids, proteins, peptides (other than those specifically described herein), amino acids, herbs, lipids, antioxidants, carotenoids, tocopherols, tocotrienols, phytosterols, polyphenols, bioflavonoids and/or dietary fiber.

Animal feeds, as well as most foods, may further include one or more components selected from a carbohydrate-containing substance, a protein-containing substance, and a lipid-containing substance, so the feeds, supplements or additives of the invention may contain one or more of these components, preferably a combination of all of these components.

Suitable starch-bearing feed/food components may for example be derived from grains, e.g. selected from the group consisting of corn, soybean, wheat, sorghum, barley, oat, and mixtures thereof. Examples of suitable starch-bearing substances include, but are not limited to, corn flour, ground corn, soybean flour, wheat flour, ground oat flour, wheat middlings, soybean meal, corn grit, and mixtures thereof.

Crude protein-bearing substances (not including the BSF protein extracts described herein) include for example, a fish meal, dried whey, a soybean meal, and mixture thereof can be used. Other suitable protein-bearing substances include, but are not limited to, soybean protein concentrate, soy flour, blood meal, plasma protein, dried skim milk, whey protein concentrate, canola meal, corn gluten meal, wheat gluten meal, yeast, sunflower meal, and mixtures thereof.

Suitable fat-containing substances include, but are not limited to, lard, tallow, soybean oil, lecithin, coconut oil, whey-fat blend, and mixtures thereof.

The animal feeds or feed supplement may comprise at least one palatability enhancer and a humectant, or any combination thereof.

Examples of palatability enhancers include, but are not limited to metal pyrophosphate, 2methyl furan, 2-methyl 10 pyrrole, dimethyl disulfide. Exemplary humectants include, but are not limited to propylene glycol, glycerine, corn syrup and a mineral salt.

The animal feed, feed composition or feed supplement may be suitable for any animal including for example for a companion animal, including a cat, dog, horse, rabbit or bird. In another embodiment, the feed is a aquaculture feed including, shrimp such as white leg shrimp, tuna, cod, a farmed marine organism, a farmed fresh water organism, turbot, salmon, tilapia and carp.

According to another embodiment, the animal is a livestock food and further comprises plant biomass (e.g. vegetable biomass).

Non-limiting examples of suitable livestock animals may include pigs, cows, horses, goats, sheep, llamas and alpacas.

It will be appreciated that since the protein/peptide extract described herein is contemplated for use as a feed or a feed (or food) additive, the present invention further comprises methods of protecting the peptides from the harsh conditions of the gastrointestinal (GI) tract including the stomach, mouth, esophagus and rumen. Additionally, or alternatively, the BSF protein extract may be formulated for oral delivery so as to allow for controlled/sustained release in the intestines, or to increase their half-life. Such methods include for example encapsulation, lipidization, cationization and PEGylation of the protein/peptides, all of which are known to one skilled in the art.

The amount of BSF protein extract per capsule is typically between 1-65%.

Final concentration of BSF protein extract in the feeds are between 1-2500 ppm, e.g. 10-1000 ppm or 25-750 ppm.

In one embodiment, proteins obtained from the dry protein extraction process are solubilized in water/buffer and maintained in liquid until encapsulation or microencapsulation.

In another embodiment, proteins obtained from the dry protein extraction process are dried and optionally stored as powder. The powder is then reconstituted into a liquid prior to encapsulation or microencapsulation.

In still another embodiment, the watery fraction obtained from the wet protein extraction process is used for encapsulation or microencapsulation.

In still another embodiment, a concentrated watery fraction obtained from the wet protein extraction process is used for encapsulation or microencapsulation.

In still another embodiment, the watery fraction is dried and optionally stored as a powder. The powder may then be reconstituted into a liquid prior to encapsulation or microencapsulation.

In still another embodiment, the solid mass obtained from the wet protein extraction process may be rinsed in order to obtain a soluble fraction of the protein pulp (and optionally concentrated) and optionally used for encapsulation or microencapsulation.

In still another embodiment, the solid mass obtained from the wet protein extraction process may be rinsed in order to obtain a soluble fraction of the protein pulp, dried and optionally stored as a powder. The powder is then reconstituted into a liquid prior to encapsulation or microencapsulation.

In still another embodiment, the solid mass obtained from the wet protein extraction process is dried and subsequently soluble proteins are extracted using water or buffer to produce a soluble fraction which can be used directly, or dried or concentrated prior to encapsulation or microencapsulation.

In still another embodiment, the solid mass obtained from the wet protein extraction process is added to the sticky water fraction (with or without the additional of additional solvent) prior to encapsulation or microencapsulation.

Natural polymers that may be used for encapsulation or microencapsulation include but are not limited to sodium alginate, gum Arabic, chitosan, cellulose and carboxymethylcellulose, pectin, Shellac, xanthan gum, gum Arabic, zein, pullulan, maltodextrin, whey protein, galactomannan, modified starch, polycaprolactone, cyclodextrin, carrageenan, casein and sodium caseinate.

Exemplary methods for encapsulating the proteins/peptides include freeze-drying, spray-drying, spray-chilling, fluidized bed, extrusion, coacervation, complexation, and supercritical anti-solvent drying.

The encapsulation may result in monolayer capsules, multilayer capsules, multinuclear capsules, regular shaped capsules, matrices or irregular shaped capsules.

In one embodiment, the proteins/peptides are added to a mineral carrier, such as Attapulgite or Diatomaceous powder. The particle diameter of the carrier mineral can be between 1 micron to 2,000 microns. Loading % may range between 1%-60%.

In another embodiment, the proteins/peptides are loaded on a feed-grade nutritional carrier, such as, but not limited to: grains powder, wheat powder, rice powder, Zaine powder etc. The particle diameter of the nutritional carrier can be between 1 micron to 2,000 microns. Loading % may range between 1%-60%.

The encapsulated or non-encapsulated proteins/peptides may be comprised in conventional pellets (for layers, broilers, swine, calves, cows and companion animals) and into extruded pellets for aquaculture. In addition, the encapsulated or non-encapsulated proteins/peptides may be added to powdered (e.g. not pelleted or extruded) feed used for an array of livestock animals and aquaculture. In addition, the encapsulated or non-encapsulated proteins/peptides may be added to liquids (e.g. drinking water) used for an array of livestock animals.

The feed may further comprise an absorption enhancer and/or a mucolytic agent.

The present inventors have surprisingly found that protein/peptide extracts described herein show anti-inflammatory properties and accordingly can be used for preventing and/or treating inflammatory diseases of animals and for suppressing pro-inflammatory responses.

Examples of inflammatory diseases include inflammatory bowel diseases (IBDs), which are severe gastrointestinal disorders characterized by intestinal inflammation and tissue remodeling, that increase in frequency and may prove disabling for patients. The major forms of IBD, ulcerative colitis (UC), Celiac and Crohn's disease are chronic, relapsing conditions that are clinically characterized by abdominal pain, diarrhea, rectal bleeding, and fever.

Thus, according to another aspect of the present invention, there is provided a method of treating or preventing an inflammatory disease or inflammatory conditions in a subject comprising providing a therapeutically effective amount of the animal feed or human food described herein, the BSF protein extract described herein or the hydrolyzed BSF protein extract described herein to the subject, thereby treating or preventing the inflammatory disease of the subject.

As used herein, the term “subject” includes mammals, including human beings, companion animals, livestock animals, at any age which suffer from the pathology. Those in need of treatment may include individuals already having IBD, as well as those at risk of having, or who may ultimately acquire the disease. The need for treatment is assessed, e.g., by the presence of one or more risk factors associated with the development of IBD, the presence or progression of IBD, or likely receptiveness to treatment of a subject having IBD. For example, “treating” IBD may encompass reducing or eliminating associated symptoms, and does not necessarily encompass the elimination of the underlying disease etiology, e.g., a genetic instability locus.

In some embodiments, a subject with IBD is in remission and/or presently asymptomatic, and the BSF protein/peptide extract described herein may be administered during the remission period to reduce the potential for a flare-up. In some embodiments, the subject in remission and/or presently asymptomatic for IBD is undergoing treatment, e.g., antibiotics and/or steroids, and the BSF protein/peptide extract described herein may be co-administered with such treatment to reduce the potential for a flare-up.

The BSF proteins/peptides can be provided to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the BSF proteins/peptides accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to the composition to further facilitate administration of an active ingredient. Examples, without limitation, of excipients include calcium carbonate, calcium phosphate, various sugars, such as glucose, glucosamine and N-acetyl-glucosamine, and types of oligo or polysaccharides such as starch, cellulose derivatives, gelatin, chitin, chitosan, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal, pulmonary or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., inflammatory disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing healthcare professional, such as a physician or a vet, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In some embodiments, the term “about” refers to ±20% or ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques.

### Example 1

### Materials and Methods

BSF meal preparation 1: A commercially available preparation was used (www(dot)zmf(dot)co(dot)il/).

BSF meal preparation 2:10-14 day BSF larvae were collected in-house and separated from left-over frass. Additional processing was also carried out in-house as further described below. The larvae were blanched in boiling water for 3 minutes and subsequently dried in a drying oven until humidity reached 3%. Subsequently, the larvae were placed into an expeller in order to separate between the oil fraction and dry meal fraction.

Assessment of anti-inflammatory activity: the effect of a sample on the secretion of key cytokines from human peripheral blood mononuclear cells (PBMC) following stimulus was determined. PBMC were collected from venous blood, washed in phosphate buffered saline and incubated in culture medium. The cells were supplemented with either the sample, the vehicle (the solvent the sample was in) or dexamethasone. Following incubation, the cells were challenged with phytohemagglutinin to induce an inflammatory response. Secretion of cytokines was followed by ELISA analysis (IL6: R&D cat #D206; IL1b: R&D cat #DY201; TNFα: R&D cat #DY210).

Protein solubilization optimization: BSF protein meal was solubilized in 100 mM Tris pH7.5, at 5% w/v, 2.5% w/v, 1% w/v, 0.5% w/v, 0.25% w/v, 0.1% w/v and 0.05% w/v. The mixture was vortexed and then centrifuged at 4000 g for 20 minutes at room temperature. The supernatant was collected, and the presence of proteins was estimated by the change in absorbance at 280 nm (Nanovue Plus spectrophotometer, GE). Several physical conditions were tested: vortexing, continual mixing at room temperature for 1 hour, incubation at 37 °C for 1 hour and at 50 °C for 1 hour. A second experiment testing BSF meal:buffer ratio was performed at higher concentrations: 2.5% w/v, 5% w/v, 10% w/v and 25% w/v. Samples were stirred constantly for 1 hour at room temperature.

Comparison of 20% w/v Solubilized BSF Protein Extract with Total BSF Protein Extract:

The 20% w/v solubilized BSF protein in 100 mM Tris-HCl pH 7.5 was prepared as described above. The total BSF protein extract was prepared by solubilizing 1 gr of defatted BSF protein meal in 10% SDS to a final volume of 10 ml.

75 μl of each of the protein extracts was transferred to 1.5 ml centrifuge tubes. To each tube, 25 μl 4× Laemmli sample buffer was added to which was supplemented 10 μl 1M DTT. The samples were incubated for 1 hour at room temperature and separated on a 4-20% Precast Protein Gel. Following fixation, the gel was stained for at least 1 hour with Coomassie stain, washed with water and analyzed.

BSF digestion by protease: Four proteases were tested: trypsin, Aspartate-N (Asp-N), Glutamate-C (Glu-C) and ubiquitin specific peptidase 5 (UDP5, all from Sigma Aldrich). Protein breakdown was tested for the different enzymes, 2.5 μl per 100 μl 20% BSF protein solution at 37 °C for 1, 2, 4 hours and overnight. 25 μl of 5× reducing protein sample buffer were added to the protein samples, and they were incubated at 80 °C for 20 minutes and then spun down at maximum speed for 5 minutes on a benchtop centrifuge.

BLAST-P: Two AMP datasets were aligned with BSF proteins using BLAST-P (Altschul S F, 1990) with default scoring parameters: BLOSUM62 matrix, gap existence cost=11, gap extension cost=1. Then, alignment results were filtered to allow no mismatches (i.e., 100% identity) and a maximum of 5 target sequences per target protein.

Expression: Transcriptomic Data was retrieved from the European Nucleotide Center (ENA)—Study Accession: PRJNA506627. The 24 Samples of 150 bp pair-end reads were evaluated for read quality by fastQC (v0.12.1). Reads were aligned to the reference BSF genome (NCBI RefSeq sequence: GCF_905115235.1, (Generalovic T A, 2021)) by STAR aligner (v2.7.10a (Dobin A, 2013)). An average of ~83.6% of the reads were aligned and ~68.2% of them were assigned to genes by HTseq-count (v1.99.2). Differential expression patterns and normalized counts matrix were retrieved by DEseq2 (Love M I, 2014).

Identifying digestion site: Final trapped-peptides candidates were analyzed for peptide cleavage sites. Relevant commercial peptidase, beneficial for “rescuing” these trapped-peptides were identified. For each cleavage site, four prior and subsequent amino acids were inserted to the MEROPS database (Rawlings N D, 2018), and relevant results were integrated.

### Results

Two publicly available AMP datasets, ADP3 (Wang G, 2016) and DRAMP (Shi G, 2022) were obtained. They were aligned with the BSF proteins dataset generated from the BSF genome (Generalovic T A, 2021) using BLAST-P (Altschul S F, 1990) with the standard scoring system. Then, alignment results were filtered to allow no mismatches (i.e., 100% identity) and a maximum of 5 sequences per target protein. Identified AMPs are presented in Table 3, herein below. The identified peptides are found within the amino acid sequence of native BSF proteins and are referred to herein as “trapped peptides”. Known anti-microbial peptide encoding genes such as DLPs and CLPs were filtered out.

TABLE 3

| | SEQ |
|---|---|
| | ID |
| Peptide sequence | NO: |
| MQIFVKTLTGKTITLEVEPSDTIENVKAKIQDKEGIPPD | 1 |
| QQRLIFAGKQLEDGRTLSDYNIQKESTLHLVLRLR | |
| VALTGLTVAEYFR | 2 |
| ARTKQTARKSTGGKAPRKQLAT | 3 |
| RGFRKHFNNLPICVEGLAGDIGSILLGVGSDIGALAGAI | 4 |
| ANLALIAGECAAQGEAGAAVVAAT | |
| MASKFLGNPNHNIGGGVFAAGNTRSNTPSLGAFGTLNLK | 5 |
| DHSLGVSHTITPGVSDTFSQNARLNILKTPDHRVDANVF | |
| NSHTRLNNGFAFDKRGGSLDYTHRAGHGLSLGASHIPKF | |
| GTTAELTGKANLWRSPSGLSTFDLTGSASRTFGGPMAGR | |
| NNFGAGLGFSHRF | |
| MWITNGGVANWYFVLAR | 6 |
| AERVGAGAPVYL | 7 |
| MAGGKAGKDSGKAKAKAVSRSARAGLQFPVGRIHRHLK | 8 |
| MTGRGKGGKGLGKGGAKRHRKVLRDNIQGITKPAIRRLA | 9 |
| RRGGVKRISGLIYEETRGVLKVFLENVIRDAVTYTEHAK | |
| RKTVTAMDVVYALKRQGRTLYGFGG | |
| WKWKWK | 10 |
| CHPSTHRYC | 11 |
| KGLSGPSEWWVWV | 12 |
| KVHGSLARAGK | 13 |
| RQIKIWFQNRRMKWKK | 14 |
| EPRTPWDWV | 15 |

### Release of Potential of BSF “Trapped Peptides”

As mentioned above, the identified peptides appear within larger proteins. To exert their biologically active, these peptides should be released from their parent proteins. To “free” such a peptide, the parent protein must be hydrolyzed at the peptide's N and C termini. Peptide bond hydrolysis in general, and at specific locations in particular, can be done by proteases. Several proteases are known to hydrolyze peptide bonds prior to or following certain amino acids or amino acid sequences. For example, trypsin hydrolyzes the peptide bonds following positively charged amino acids: lysine and arginine. The amino acid sequences of the parent proteins of the disclosed target “trapped peptides” were analyzed using MEROPS (Rawlings N D, 2018). The candidate proteins were individually checked for the presence of specific proteases (i.e., proteases with at most two specific amino acids or sequences digestion site recognition). Specific proteases were identified for three “trapped peptides” found within 7 proteins (Table 4). One peptide, VK10 (SEQ ID NO: 1), can be released from its parent protein, histone H3.3A, by cathepsin L, or by trypsin (adding an extra lysin to the peptide). Another peptide, sarconesin (SEQ ID NO: 2), can be released from its parent protein, mitochondrial ATP synthase beta subunit, by a combination of trypsin and arginine-C protease. The third peptide, cgUbiquitin (SEQ ID NO: 3), can be released from its parent proteins, different ubiquitins and poly-ubiquitins, by ubiquitin specific peptidase 5 (USP5), 4 or 15.

TABLE 4

| | | | length | # | Specific |
|---|---|---|---|---|---|
| Gene | Description | peptide | (aa) | repeats | peptidase |
| LOC119656929 | histone H3.3A | VK10 | 22 | 1 | cathepsin |
| LOC119647429 | histone H3.3A | (SEQ ID | | 1 | L/trypsin |
| | | NO: 1) | | | |
| LOC119660807 | ATP synthase | Sarconesin | 13 | 1 | Trypsin |
| | subunit beta, | (SEQ ID | | | Arg-C |
| | mitochondrial | NO: 2) | | | proteinase |
| LOC119653258 | ubiquitin-60S | cgUbiquitin | 74 | 1 | OTULIN |
| | ribosomal | (SEQ ID | | | peptidase |
| | protein L40 | NO: 3) | | | ubiquitinyl |
| LOC119650658 | ubiquitin-40S | | | 1 | hydrolase- |
| | ribosomal | | | | L3 |
| | protein S27a | | | | ubiquitin- |
| LOC119658576 | polyubiquitin- | | | 9 | specific |
| | C | | | | peptidase 5 |
| LOC119655225 | polyubiquitin- | | | 12 | ubiquitin- |
| | C-like | | | | specific |
| | | | | | peptidase 4 |
| | | | | | ubiquitin- |
| | | | | | specific |
| | | | | | peptidase 15 |

### BSF Meal Protein Solubilization

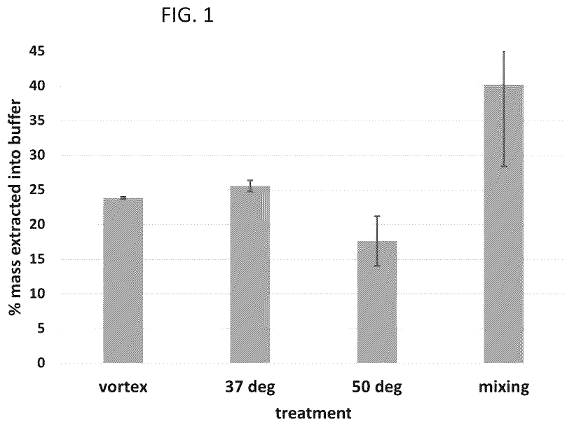

To optimize solubilization, several physical conditions and several BSF meal to buffer ratios were tested. Two different BSF meals were analyzed (as denoted in materials and methods), To separate the solubilized proteins from the insolubilized matter, the samples were centrifuged, and the supernatant was collected. The amount of solubilized protein was estimated by the change in absorbance at 280 nm. It was found that stirring the BSF meal for 1 hour at room temperature extracted proteins better than the other methods tested (FIG. 1). Increasing BSF meal:buffer ratios up to 20% (w/v) BSF meal, resulted in a linear increase in the absorbance at 280 nm, indicating an increased extraction of proteins into the buffer (Table 5).

TABLE 5

| % BSF meal in | OD | |
|---|---|---|
| solution | 280 nm | % extracted |
| 2.5 | 0.233 | 85.44 |
| 5 | 0.427 | 86.67 |
| 10 | 0.8 | 87.43 |
| 25 | 1.568 | 84.59 |

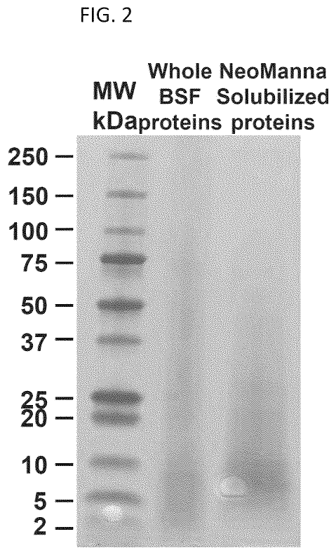

A 20% w/v solubilized BSF protein extract was compared with total BSF protein extract using SDS-PAGE. As seen in FIG. 2, the 20% solubilized extract is enriched in small proteins and peptides compared to total BSF protein extract.

### Example 2

### Wet Process for Generating BSF Meal

### Materials and Methods

BSF meal preparation: 10 day larvae were collected in-house and washed under tap water. Additional processing was also carried out in-house as further described below. The larvae were microwaved for 5 minutes and strained using a sieve. Distilled water was added. The larvae were then ground using a blender and the pulp heated for one hour at 88 °C. Following centrifugation and overnight storage at 4 °C, three fractions were obtained. The top layer comprised the fat fraction. The middle layer comprised soluble proteins (also referred to herein as sticky water). The bottom layer (also referred to herein as the solid pulp fraction) also comprised soluble proteins.

Meal of the different fractions was made by:
(i) drying the watery fraction alone;
(ii) drying solid pulp fraction; and
(iii) mixing the watery fraction with the solid pulp fraction together and drying said mix.
   The sticky water fraction was also tested as is without drying.

### Example 3

### Anti-Inflammatory Effect of Solubilized BSF Extract

BSF solubilization: Protein extraction was carried out by combining 20% BSF meal in 100 mM tris-HCl pH 7.5 at room temperature for 1 hour. The reaction was dried by lyophilization. The resulting pellet was dissolved to 10% in DMSO.

Assay for measuring anti-inflammatory activity: as described in Example 1.

### Results

As illustrated in FIGS. 3A-B, solubilized BSF from meal preparation 1 leads to a reduction in the release of pro-inflammatory cytokines from PBMC cells at compared to carrier. The reduction at 10 ppm is similar to that caused by 10 ppm Dexamethasone. Reduction in secretion is stronger at 100 and 1000 ppm.

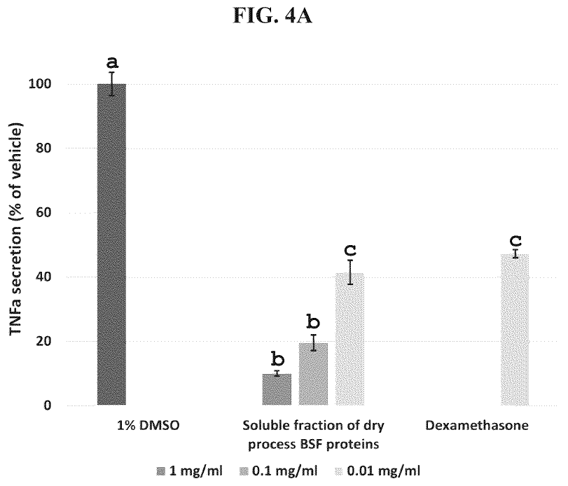

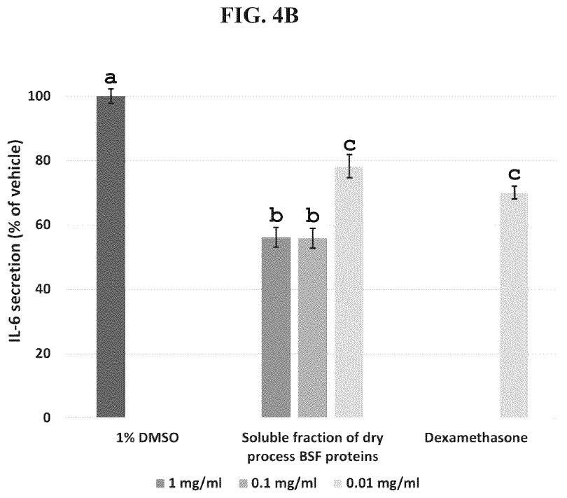

As illustrated in FIGS. 4A-B, solubilized BSF from meal preparation 2 leads to a reduction in the release of pro-inflammatory cytokines from PBMC cells at compared to carrier. The reduction at 10 ppm is similar to that caused by 10 ppm Dexamethasone. Reduction in secretion is stronger at 100 and 1000 ppm.

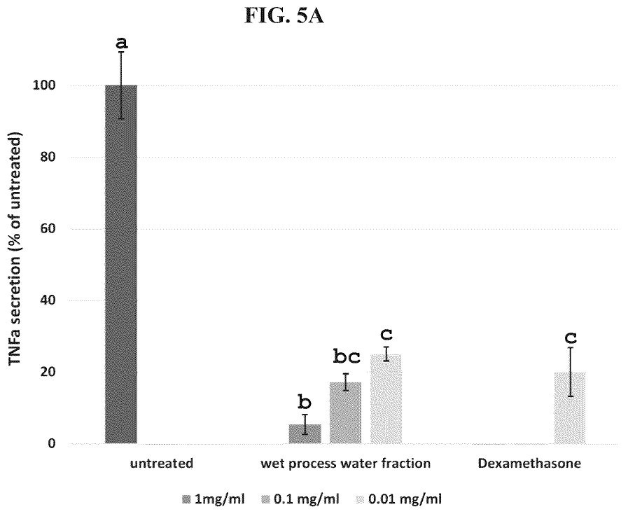

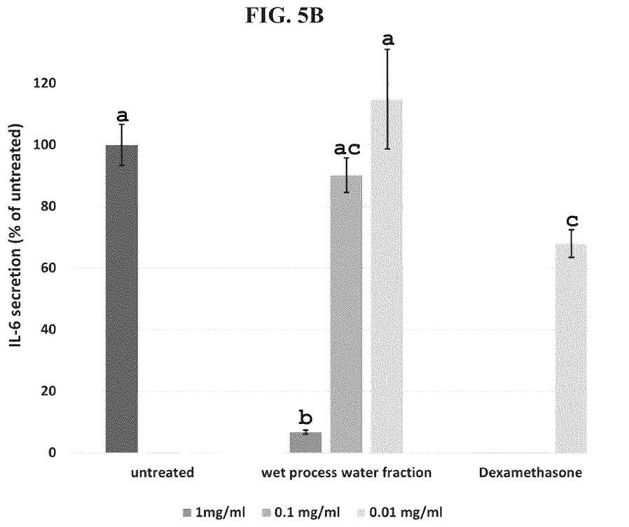

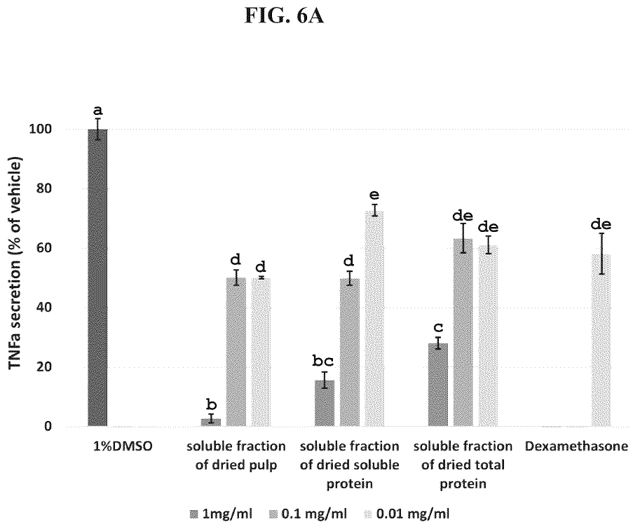

As illustrated in FIGS. 5A-B, solubilized BSF from watery fraction (sticky water) without drying leads to a reduction in the release of pro-inflammatory cytokines from PBMC cells at compared to carrier.

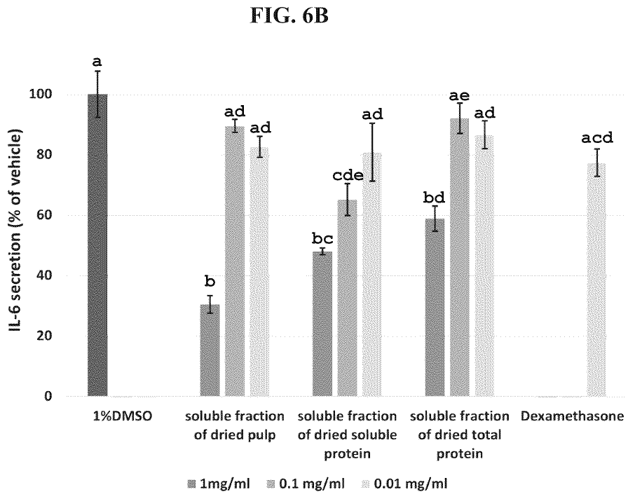

As illustrated in FIGS. 6A-B, solubilized BSF from dried fractions obtained via the wet process, dried and then re-solubilized (as described in Example 2) effect the release of pro-inflammatory cytokines from PBMC cells.

For FIGS. 3A-B-6A-B, statistical significance, designated by lower case letters, was determined by one way ANOVA followed by Tukey HSD post-hoc analysis (p<0.05).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. (canceled)

2. A black soldier fly (BSF) protein extract comprising at least one peptide consisting of the amino acid sequence selected from the group consisting of SEQ ID NO: 2-15.

3. A black soldier fly (BSF) protein extract being enriched or supplemented with at least one peptide consisting of the amino acid sequence selected from the group consisting of SEQ ID NO: 1-15.

4. The BSF protein extract of claim 2, wherein said extract is devoid of peptides of less than 6 amino acids.

5. The BSF protein extract of claim 2, wherein said at least one peptide is formulated for oral delivery.

6-13. (canceled)

14. A method of preparing a hydrolyzed BSF protein extract comprising:
contacting a protein-solubilized BSF protein extract with:
(i) a hydrolyzing enzyme selected from the group consisting of ubiquitin specific peptidase 5 (USP5), ubiquitin specific peptidase 4 (USP4) and ubiquitin specific peptidase 15 (USP15) under conditions that allow said enzyme to hydrolyze said BSF extract;
(ii) a combination of trypsin and arginine-C protease under conditions that allow said trypsin and arginine-C protease to hydrolyze said BSF extract, thereby preparing the hydrolyzed BSF protein extract; or
(iii) Glu-N protease under conditions that allow said Glu-N protease to hydrolyze said BSF extract.

15. The method of claim 14, wherein said protein-solubilized BSF protein extract is generated from BSF larvae.

16. The method of claim 15, wherein said larvae are between 2-40 days of age post hatching.

17. The method of claim 14, further comprising generating said protein-solubilized BSF protein extract by:
(a) defatting BSF or BSF larvae thereof to generate a defatted extract of BSF or BSF larvae;
(b) solubilizing said defatted extract.

18. The method of claim 17, wherein said solubilizing is effect in a buffer, a solvent or water.

19. The method of claim 18, wherein said buffer comprises Tris-HCl.

20. The method of claim 17, wherein said BSF or BSF larvae are dried prior to defatting.

21. The method of claim 17, further comprising generating a pulp from said BSF or BSF larvae prior to said defatting.

22. The method of claim 21, further comprising separating a water-soluble fraction of the pulp from solid material of the pulp following said defatting.

23. The method of claim 21, further comprising drying said pulp prior to said solubilizing.

24. The method of claim 17, wherein said defatting is effected by at least one of the separating means selected from the group consisting of decantation, tricantation, centrifugation and pressing.

25. A hydrolyzed BSF protein extract obtainable according to the method of claim 14.

26-39. (canceled)

40. A method of treating or preventing an inflammatory disease in a subject comprising providing a therapeutically effective amount of the BSF protein extract to the subject, thereby treating or preventing the inflammatory disease of the subject.

41. (canceled)

42. The method of claim 40, wherein said BSF protein extract is obtained by removing lipids from BSF larvae or BSFs under wet conditions.

43. The method of claim 40, wherein said BSF protein extract is obtained by removing lipids from BSF larvae or BSFs under dry conditions.

44-47. (canceled)

48. The method of claim 40, wherein said inflammatory disease is IBD, Celiac, Crohn's disease or Colitis.
